(19) United States
(12) Patent Application Publication
(10) Pub. No.: US 2017/0072198 A1
(43) Pub. Date: Mar. 16, 2017

(54) METHODS TO CONCURRENTLY STIMULATE DIFFERENT BRAIN TARGETS

(71) Applicant: Boston Scientific Neurornodulation Corporation, Valencia, CA (US)

(72) Inventors: James C. Makous, N. Potomac, MD (US); Courtney C. Lane, Ventura, CA (US); Kristen Jaax, Santa Clarita, CA (US); Andrew DiGiore, San Francisco, CA (US)

(21) Appl. No.: 15/359,164

(22) Filed: Nov. 22, 2016

Related U.S. Application Data
(63) Continuation of application No. 12/976,764, filed on Dec. 22, 2010, now abandoned.
(60) Provisional application No. 61/290,427, filed on Dec. 28, 2009.

(51) Int. Cl. A61N 1/36; A61N 1/05

(52) U.S. Cl. CPC A61N 1/36067; A61N 1/0534; A61N 1/36064; A61N 1/36171

(57) ABSTRACT

A method for treating a patient having a dysfunction using a stimulation lead within the brain of a patient is provided. The stimulation lead carries a plurality of electrodes adjacent to a plurality of brain regions. Pulsed electrical waveforms having different sets of stimulation parameters are generated and then concurrently delivered to the plurality of electrodes, thereby concurrently stimulating the plurality of brain regions to treat the dysfunction.

### RELATED APPLICATION

The present application claims the benefit under 35 U.S.C. §119 to U.S. provisional patent application Ser. No. 61/290,427, filed Dec. 28, 2009. The foregoing application is hereby incorporated by reference into the present application in its entirety.

### FIELD OF THE INVENTION

The present invention relates to methods of neurostimulation, and more particularly, to methods for treating disorders via brain stimulation.

### BACKGROUND OF THE INVENTION

Implantable neurostimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) systems have long been accepted as a therapeutic modality for the treatment of chronic pain syndromes, and the application of tissue stimulation has begun to expand to additional applications such as angina pectoralis and incontinence.

More pertinent to the present inventions described herein, Deep Brain Stimulation (DBS) has been applied therapeutically for well over a decade for the treatment of neurological disorders. DBS and other related procedures involving implantation of electrical stimulation leads within the brain of a patient are increasingly used to treat disorders, such as Parkinson's disease, essential tremor, seizure disorders, obesity, depression, obsessive-compulsive disorder, Tourette's syndrome, dystonia, and other debilitating diseases via electrical stimulation of one or more target sites, including the ventrolateral thalamus, internal segment of globus pallidus, substantia nigra pars reticulate, subthalamic nucleus (STN), or external segment of globus pallidus. DBS has become a prominent treatment option for many disorders, because it is a safe, reversible alternative to lesioning. For example, DBS is the most frequently performed surgical disorder for the treatment of advanced Parkinson's disease. There have been approximately 30,000 patients world-wide that have undergone DBS surgery. Consequently, there is a large population of patients who will benefit from advances in DBS treatment options. Further details discussing the treatment of diseases using DBS are disclosed in U.S. Pat. Nos. 6,845,267 and 6,950,707, which are expressly incorporated herein by reference.

Implantable neurostimulation systems typically include one or more electrode carrying stimulation leads, which are implanted at the desired stimulation site, and a neurostimulator (e.g., an implantable pulse generator (IPG)) implanted remotely from the stimulation site, but coupled either directly to the stimulation lead(s) or indirectly to the stimulation lead(s) via a lead extension. The neurostimulation system may further comprise an external control device to remotely instruct the neurostimulator to generate electrical stimulation pulses in accordance with selected stimulation parameters.

Electrical stimulation energy may be delivered from the neurostimulator to the electrodes in the form of a pulsed electrical waveform. Thus, stimulation energy may be controllably delivered to the electrodes to stimulate neural tissue. The combination of electrodes used to deliver electrical pulses to the targeted tissue constitutes an electrode combination, with the electrodes capable of being selectively programmed to act as anodes (positive), cathodes (negative), or left off (zero). In other words, an electrode combination represents the polarity being positive, negative, or zero. Other parameters that may be controlled or varied include the amplitude, duration, and frequency of the electrical pulses provided through the electrode array. Each electrode combination, along with the electrical pulse parameters, can be referred to as a “stimulation parameter set.”

With some neurostimulation systems, and in particular, those with independently controlled current or voltage sources, the distribution of the current to the electrodes (including the case of the neurostimulator, which may act as an electrode) may be varied such that the current is supplied via numerous different electrode configurations. In different configurations, the electrodes may provide current or voltage in different relative percentages of positive and negative current or voltage to create different electrical current distributions (i.e., fractionalized electrode configurations).

As briefly discussed above, an external control device can be used to instruct the neurostimulator to generate electrical stimulation pulses in accordance with the selected stimulation parameters. Typically, the stimulation parameters programmed into the neurostimulator can be adjusted by manipulating controls on the external control device to modify the electrical stimulation provided by the neurostimulator system to the patient. However, the number of electrodes available combined with the ability to generate a variety of complex stimulation pulses, presents a vast selection of stimulation parameter sets to the clinician or patient.

To facilitate such selection, the clinician generally programs the neurostimulator through a computerized programming system. This programming system can be a self-contained hardware/software system, or can be defined predominantly by software running on a standard personal computer (PC). The PC or custom hardware may actively control the characteristics of the electrical stimulation generated by the neurostimulator to allow the optimum stimulation parameters to be determined based on patient feedback or other means and to subsequently program the neurostimulator with the optimum stimulation parameter set or sets, which will typically be those that stimulate all of the target tissue in order to provide the therapeutic benefit, yet minimizes the volume of non-target tissue that is stimulated. The computerized programming system may be operated by a clinician attending the patient in several scenarios.

In the context of DBS, a multitude of brain regions may need to be electrically stimulated in order to treat one or more ailments associated with these brain regions. To this end, multiple stimulation leads are typically implanted adjacent the multiple brain regions. In particular, multiple burr holes are cut through the patient's cranium as not to damage the brain tissue below, a large stereotactic targeting apparatus is mounted to the patient's cranium, and a cannula is scrupulously positioned through each burr hole one at a time towards each target site in the brain. Microelectrode recordings may typically be made to determine if each trajectory passes through the desired part of the brain, and if so, the stimulation leads are then introduced through the cannula, through the burr holes, and along the trajectories into the parenchyma of the brain, such that the electrodes located on the lead are strategically placed at the target sites in the brain of the patient.

Disadvantageously, the cutting of multiple burr holes and the introduction of the leads along multiple trajectories into the brain increases trauma and risk to the patient.

Furthermore, stimulation of multiple brain regions with sets of stimulation parameters has been shown to be useful. For example, stimulation of the pedunculopontine (PPN) and subthalamic nuclei (STN) at different frequencies has been shown to be beneficial (see Alessandro Stefani, et al. “Bilateral Deep Brain Stimulation of the Pedunculopontine and Subthalamic Nuclei in Severe Parkinson's Disease,” Brain (2007); 130 1596-1607). In another DBS example, one frequency is used to optimize treatment of tremor and rigidity, while another frequency is used to treat bradykinesia (see U.S. Pat. No. 7,353,064).

Thus, if the same set of stimulation parameters is used to stimulate the different brain regions, either (1) one brain region may receive optimal therapy and the other brain region may receive poor therapy, or, (2) both brain regions may receive mediocre therapy. Thus, to maximize the therapeutic effects of DBS, each brain region may require different sets of stimulation parameters (i.e. different amplitudes, different durations, and/or frequencies).

One way that prior art DBS techniques attempt to stimulate several brain regions using different stimulation parameters is to implant multiple leads adjacent the different regions of the brain, and quickly cycling the stimulation through the brain regions with the different stimulation parameters. In some applications, such as the treatment of chronic pain, this effect may be unnoticeable; however, the brain is a complex system of rapidly transmitting electric signals, and the effect of rapid cycling may produce a “helicopter effect” that may undesirably result in ineffective treatment and/or side-effects such as seizures.

Another way that prior art DBS techniques attempt to stimulate several brain regions using different stimulation parameters is to connect the multiple leads to multiple neurostimulators respectively programmed with different stimulation parameters.

Thus, there remains a need to provide an improved method for concurrently stimulating multiple brain regions with different sets of stimulation parameters.

### SUMMARY OF THE INVENTION

In accordance with one embodiment of the present invention, a method for treating a patient having a dysfunction using a stimulation lead implanted within the brain of the patient, the implanted lead carrying a plurality of electrodes (e.g. a single lead with a plurality of in line electrodes) located adjacent a respective plurality of regions of the brain. The method includes the steps of (i) generating a plurality of pulsed electrical waveforms, each pulsed electrical waveform having a different set of stimulation parameters (e.g. different frequencies or different pulse durations), and (ii) concurrently delivering the plurality of pulsed electrical waveforms respectively to the plurality of electrodes, thereby stimulating the plurality of brain regions to treat the dysfunction.

In one method, the dysfunction may be Parkinson's disease, in which case, the plurality of stimulated brain regions may be a ventralis intermedius and a subthalamic nucleus, or the pedunculopontine and the subthalamic nucleus, or Zona incerta fibers and the subthalamic nucleus. In another method, the dysfunction may be epilepsy, in which case, the plurality of stimulated brain regions may be an anterior nucleus of the thalamus and a subthalamic nucleus. In another method, the plurality of stimulated brain regions may be a region that is formed by nerve cell bodies, and a region that is formed by nerve fibers.

In another method, the implantable stimulation lead is coupled to a single implantable pulse generator, in which case the method further comprises programming the implantable pulse generator with the different stimulation parameter sets. Still another method further comprises implanting the stimulation lead within the brain of the subject.

Although the present inventions should not be so limited in their broadest aspects, the method of concurrent delivery of a plurality of waveforms, each waveform having a different set of stimulation parameters targeted to different regions of the brain, has the advantage of reducing the number of implantable brain leads. Further advantages include reducing the costs, length, and risk of surgery by using only a single implantable stimulation lead with the capability of stimulating different brain regions with a different set of parameters for each region. A further advantage includes eliminating the “helicopter” effect by concurrently stimulating a plurality of brain regions instead of the cycling the electrical waveform to a plurality of brain regions.

Other and further features and advantages of embodiments of the invention will become apparent from the following detailed description, when read in view of the accompanying figures.

### BRIEF DESCRIPTION OF DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of an embodiment of a deep brain stimulation (DBS) system arranged in accordance with the present inventions;

FIG. 2 is a profile view of an implantable pulse generator (IPG) and percutaneous lead used in the DBS system of FIG. 1;

FIG. 3 is a timing waveform diagram that depicts representative current waveforms that may be applied to various electrode contacts of the electrode arrays through one or more stimulus channels;

FIG. 4 is a plan view of the DBS system of in use with a patient; and

FIG. 5 is a frontal cross-sectional view of a patient's head showing the implantation of a stimulation lead in contact with a patient's thalamus and subthalamic nucleus.

### DETAILED DESCRIPTION OF THE EMBODIMENTS

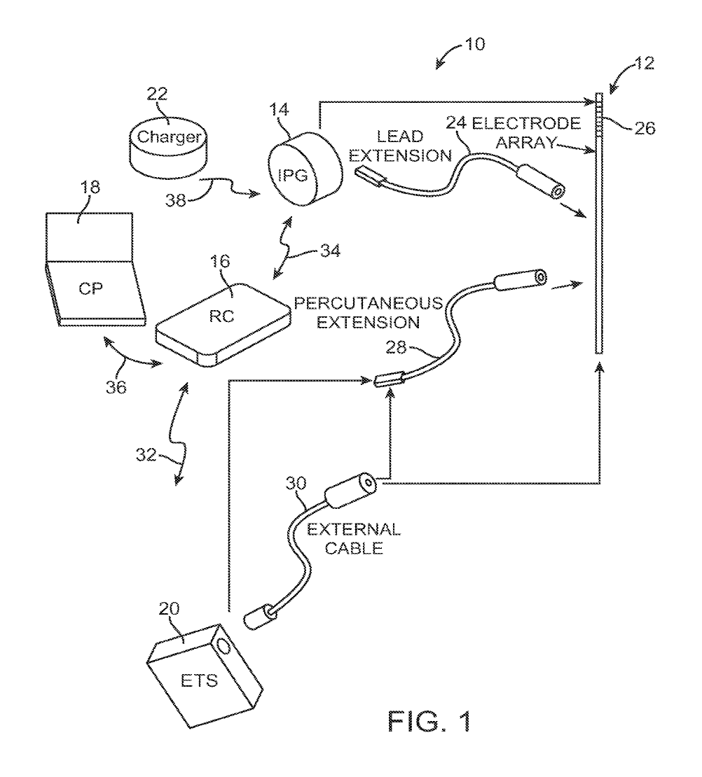

Turning first to FIG. 1, an exemplary DBS neurostimulation system 10 in accordance with one embodiment of the invention includes one implantable stimulation lead 12, an implantable pulse generator (IPG) 14, an external remote controller RC 16, a clinician's programmer (CP) 18, an External Trial Stimulator (ETS) 20, and an external charger 22.

The IPG 14 is physically connected via one percutaneous lead extension 24 to the stimulation lead 12, which carries a plurality of electrodes 26 arranged in an array. In the illustrated embodiment, the stimulation lead 12 is percutaneous lead, and to this end, the electrodes 26 may be arranged in-line along the stimulation lead 12. As will be described in further detail below, the IPG 14 includes pulse generation circuitry that delivers electrical stimulation energy in the form of a pulsed electrical waveform (i.e., a temporal series of electrical pulses) to the electrode array 26 in accordance with a set of stimulation parameters.

The ETS 20 may also be physically connected via the percutaneous lead extension 28 and external cable 30 to the stimulation lead 12. The ETS 20, which has similar pulse generation circuitry as the IPG 14, also delivers electrical stimulation energy in the form of a pulse electrical waveform to the electrode array 26 in accordance with a set of stimulation parameters. The major difference between the ETS 20 and the IPG 14 is that the ETS 20 is a non-implantable device that is used on a trial basis after the stimulation lead 12 has been implanted, and prior to implantation of the IPG 14, to test the responsiveness of the stimulation that is to be provided.

The RC 16 may be used to telemetrically control the ETS 20 via a bi-directional RF communications link 32. Once the IPG 14 and stimulation lead 12 are implanted, the RC 16 may be used to telemetrically control the IPG 14 via a bi-directional RF communications link 34. Such control allows the IPG 14 to be turned on or off and to be programmed with different stimulation parameter sets. The IPG 14 may also be operated to modify the programmed stimulation parameters to actively control the characteristics of the electrical stimulation energy output by the IPG 14.

The CP 18 may perform this function by indirectly communicating with the IPG 14 or ETS 20, through the RC 16, via an IR communications link 36. Alternatively, the CP 18 may directly communicate with the IPG 14 or ETS 20 via an RF communications link (not shown). The clinician detailed stimulation parameters provided by the CP 18 are also used to program the RC 16, so that the stimulation parameters can be subsequently modified by operation of the RC 16 in a stand-alone mode (i.e., without the assistance of the CP 18).

The external charger 22 is a portable device used to transcutaneously charge the IPG 14 via an inductive link 38. For purposes of brevity, the details of the external charger 22 will not be described herein. Once the IPG 14 has been programmed, and its power source has been charged by the external charger 22 or otherwise replenished, the IPG 14 may function as programmed without the RC 16 or CP 18 being present.

For purposes of brevity, the details of the RC 16, CP 18, ETS 20, and external charger 22 will not be described herein. Details of exemplary embodiments of these devices are disclosed in U.S. Pat. No. 6,895,280, which is expressly incorporated herein by reference.

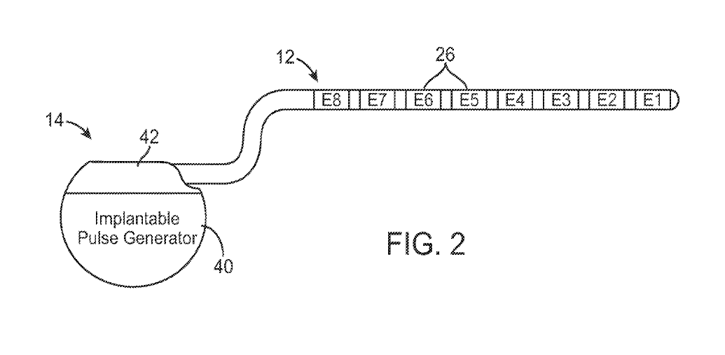

Referring now to FIG. 2, the features of the stimulation lead 12 and the IPG 14 will be briefly described. The stimulation lead 12 is shown with eight electrodes 26 (labeled E1-E8). The actual number and shape of leads and electrodes will, of course, vary according to the intended application. The IPG 14 comprises an outer case 40 for housing the electronic and other components (described in further detail below), and a connector 42 to which the proximal ends of the stimulation lead 12 mates in a manner that electrically couples the electrodes 26 to the electronics within the outer case 40. The outer case 40 is composed of an electrically conductive, biocompatible material, such as titanium, and forms a hermetically sealed compartment wherein the internal electronics are protected from the body tissue and fluids. In some cases, the outer case 40 may serve as an electrode.

As will be described in further detail below, the IPG 14 includes a battery and pulse generation circuitry that delivers the electrical stimulation energy in the form of a pulsed electrical waveform to the electrode array 26 in accordance with a set of stimulation parameters programmed into the IPG 14. Such stimulation parameters may comprise electrode combinations, which define the electrodes that are activated as anodes (positive), cathodes (negative), and turned off (zero), percentage of stimulation energy assigned to each electrode (fractionalized electrode configurations), and electrical pulse parameters, which define the pulse amplitude (measured in milliamps or volts depending on whether the IPG 14 supplies constant current or constant voltage to the electrode array 26), pulse duration (measured in microseconds), and pulse frequency (measured in pulses per second).

Electrical stimulation will occur between two (or more) activated electrodes, one of which may be the IPG case. Stimulation energy may be transmitted to the tissue in a monopolar or multipolar (e.g., bipolar, tripolar, etc.) fashion. Monopolar stimulation occurs when a selected one of the lead electrodes 26 is activated along with the case of the IPG 14, so that stimulation energy is transmitted between the selected electrode 26 and case. Bipolar stimulation occurs when two of the lead electrodes 26 are activated as anode and cathode, so that stimulation energy is transmitted between the selected electrodes 26. For example, electrode E3 on the stimulation lead 12 may be activated as an anode at the same time that electrode E4 is activated as a cathode. Tripolar stimulation occurs when three of the lead electrodes 26 are activated, two as anodes and the remaining one as a cathode, or two as cathodes and the remaining one as an anode. For example, electrodes E1 and E2 on the stimulation lead 12 may be activated as anodes at the same time that electrode E3 on the lead 12 is activated as a cathode.

The stimulation energy may be delivered between electrodes as monophasic electrical energy or multiphasic electrical energy. Monophasic electrical energy includes a series of pulses that are either all positive (anodic) or all negative (cathodic). Multiphasic electrical energy includes a series of pulses that alternate between positive and negative. For example, multiphasic electrical energy may include a series of biphasic pulses, with each biphasic pulse including a cathodic (negative) stimulation pulse and an anodic (positive) recharge pulse that is generated after the stimulation pulse to prevent direct current charge transfer through the tissue, thereby avoiding electrode degradation and cell trauma. That is, charge is conveyed through the electrode-tissue interface via current at an electrode during a stimulation period (the length of the stimulation pulse), and then pulled back off the electrode-tissue interface via an oppositely polarized current at the same electrode during a recharge period (the length of the recharge pulse). The recharge can be active (i.e. energy is expended to reverse the current) or passive (i.e. the circuit is allowed to reverse the current by connecting the circuit together in such a way that the built-up charge is discharged through the circuit).

In the illustrated embodiment, the IPG 14 can individually control the magnitude of electrical current flowing through each of the electrodes. In this case, it is preferred to have a current generator, wherein individual current-regulated amplitudes from independent current sources for each electrode may be selectively generated. Although this system is optimal to take advantage of the invention, other stimulators that may be used with the invention include stimulators having voltage regulated outputs. While individually programmable electrode amplitudes are optimal to achieve fine control, a single output source switched across electrodes may also be used, although with less fine control in programming. Mixed current and voltage regulated devices may also be used with the invention. Further details discussing the detailed structure and function of IPGs are described more fully in U.S. Pat. Nos. 6,516,227 and 6,993,384, which are expressly incorporated herein by reference.

It should be noted that rather than an IPG, the DBS neurostimulation system 10 may alternatively utilize an implantable receiver-stimulator (not shown) connected to the stimulation lead 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the stimulation in accordance with the control signals.

Significantly, the IPG 14 may be programmed by the CP 18 (or alternatively the RC 16) to operate over multiple timing channels. In particular, any combination of electrodes may be assigned to up to k possible groups, i.e., timing channels. In one embodiment, k may equal four. The timing channel identifies which electrodes are selected to synchronously source or sink current to create an electric field in the tissue to be stimulated. The programming software in the CP 18 may be used to set stimulation parameters including electrode polarity, amplitude, pulse rate and pulse duration for the electrodes of a given timing channel, among other possible programmable features. The electrode combinations assigned to the respective timing channels may be completely different from each other or can have one or more common electrodes. Thus, multiple pulsed electrical waveforms can be concurrently delivered over multiple timing channels to any of the electrodes.

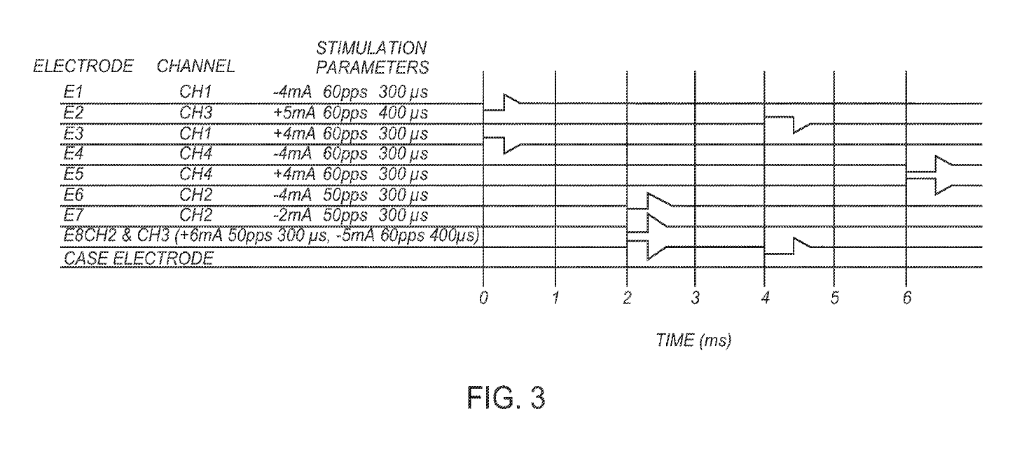

Referring to FIG. 3, one example of using four timing channels to concurrently deliver electrical pulsed waveforms to groups of the electrodes E1-E8, including the case electrode, will now be described. The horizontal axis is time, divided into increments of 1 millisecond (ms), while the vertical axis represents the amplitude of a current pulse, if any applied to one of the eight electrodes and case electrode. Thus, for example, at time t=0, channel 1 is set to generate and supply a current pulse of having a pulse amplitude of 4 (milliamps) (mA), a pulse duration of 300 microseconds (μs), and a pulse frequency of 60 pulses per second (pps) between electrode E1 (which appears as a −4 mA cathodic (negative) pulse) and E3 (which appears as a +4 mA anodic (positive) pulse). At time t=2, channel 2 is set to generate and supply a current pulse having a pulse amplitude 6 mA, a pulse duration of 300 μs, and a pulse frequency of 50 pps between electrode E8 (+6 mA) and electrodes E6 and E7 (−4 mA and −2 mA, respectively). At t=4, channel 3 is set to generate and supply a current pulse having a pulse amplitude of 5 mA, a pulse duration of 400 μs, and a pulse frequency of 60 pps between electrodes E2 (+5 mA) and electrode E8 (−5 mA). At t=6, channel 4 is set to generate and supply a current pulse having a pulse amplitude of 4 (mA), a pulse duration of 300 μs, and a pulse frequency of 60 pps between electrode E5 (+4 mA) and E4 (−4 mA).

The particular electrodes that are used with each of the channels of the IPG 14 illustrated in FIG. 3 are only exemplary of many different combinations of electrode pairing and electrode sharing that could be used. That is, any channel of the IPG 14 may be programmably connected to any grouping of the electrodes, including the reference (or case electrode). While it is typical that only two electrodes be paired together for use by a given channel of the IPG 14, as is the case with channels 1, 3, and 4, it is to be noted that any number of electrodes may be grouped and used by a given channel. When more than two electrodes are used with a given channel, the sum of the current sourced from the positive electrodes should be equal to the sum of the current sunk (returned) through the negative electrodes, as is the case with channel 2 in the example of FIG. 3 (+6 mA sourced from electrode E8, and a total of −6 mA sunk to electrodes E6 (−4 mA) and E7 (−2 mA)). It should also be appreciated that, although the individual pulses of the pulsed electrical waveforms delivered within various ones of the timing channels do not temporally overlap, the pulsed electrical waveforms delivered in these timing channels are concurrently active, and thus, considered to be concurrently delivered to the electrodes.

Further details on operating a multichannel stimulation are disclosed in the previously referenced U.S. Patent Publication No. 2007/0276450, which is expressly incorporated herein by reference.

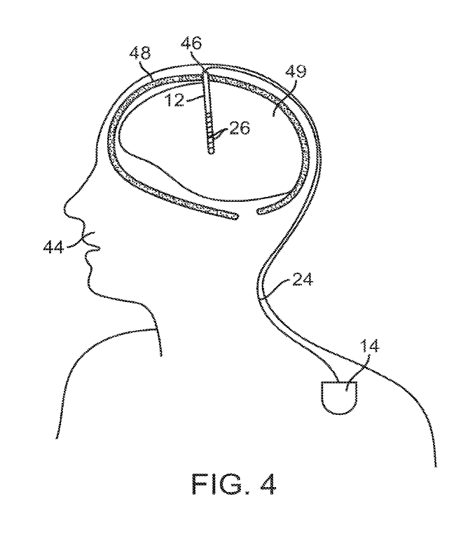

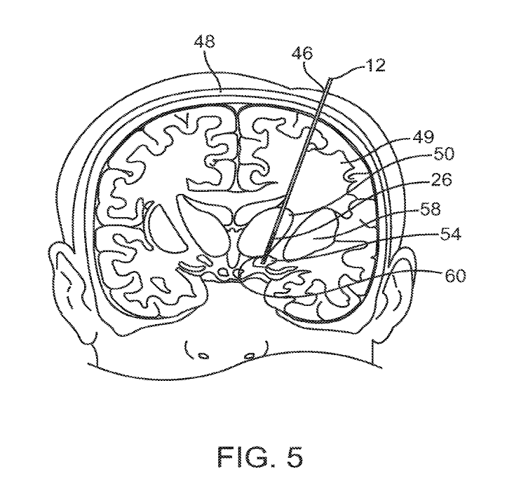

Referring now to FIGS. 4 and 5, a method of using the neurostimulation system to treat a patient will be described. The stimulation lead 12 is first introduced through a burr hole 46 formed in the cranium 48 of a patient 44, and through the parenchyma of the brain 49 of the patient 44 in a conventional manner to the thalamus 50 and subthalamic nucleus (STN) 54. Due to the lack of space near the location where the stimulation lead 12 exits the burr hole 46, the IPG 14 is generally implanted in a surgically-made pocket in the subclavicular space. The IPG 14 may, of course, also be implanted in other locations of the patient's body. The lead extension 24 facilitates locating the IPG 14 away from the exit point of the stimulation lead 12.

Significantly, the distal portion of the stimulation lead 12 carrying the electrodes 26 is long enough, so that the electrodes 26 are adjacent multiple target tissue regions whose electrical activity is the source of, or otherwise contributes to, the dysfunction or dysfunctions. Thus, stimulation energy can be conveyed from the electrodes 26 to multiple target regions to change the status of the dysfunction by concurrently delivering pulsed electrical waveforms (each defined by a different set of stimulation parameters) respectively to the electrodes 26 that are adjacent to the multiple target regions via multiple timing channels.

By way of example only, the stimulation lead 12 is situated such that the electrodes 26 are adjacent to both the subthalamic nucleus 54 and the thalamus 50, which regions can both be stimulated to treat dysfunctions, such as epilepsy and Parkinson's disease. Thus, both the STN 54 and the thalamus 50 can be concurrently stimulated using the same stimulation lead 12 by generating and concurrently delivering pulsed electrical waveforms to the specific electrodes 26 that are adjacent the STN 54 and the thalamus 50. As just discussed above, multiple timing channels can be advantageously used, so that the pulsed electrical waveforms can be concurrently delivered to these brain regions in accordance with different stimulation parameter sets in order to optimize the therapy. Notably, the thalamus 50 includes several subregions (not shown), such as the ventralis intermedius (VIM) and the anterior nucleus (AN), that can be stimulated to treat epilepsy or Parkinson's disease.

For example, to treat epilepsy, the electrodes 26 on the stimulation lead 12 are placed adjacent to the subthalamic nucleus 54 and the anterior nucleus (AN) of the thalamus 50. For optimal treatment of epilepsy, the AN of the thalamus 50 and the STN 54 are concurrently stimulated with different electrodes 26 on the same stimulation lead 12 using different stimulation parameter sets. In one exemplary method, the STN 54 is stimulated with a pulsed electrical waveform having a frequency in the range of 130 Hz-185 Hz and a pulse duration in the range of 60 μs-90 μs, while the AN of the thalamus 50 is stimulated with a pulsed electrical waveform having a frequency of 145 Hz and a pulse duration of 90 μs.

To treat Parkinson's disease, the electrodes 26 on the stimulation lead 12 are placed adjacent to the VIM of the thalamus 50 and the STN 54. For optimal treatment of Parkinson's disease, the VIM of thalamus 50 and the STN 54 are concurrently stimulated with different electrodes 26 on the same stimulation lead 12 using different stimulation parameter sets. In one exemplary method, the STN 54 is stimulated with a pulsed electrical waveform having a frequency in the range of 130 Hz-185 Hz and a pulse duration 60 μs-90 μs, while the VIM of the thalamus 50 is stimulated with a pulsed electrical waveform having a frequency in the range of 133 Hz-188 Hz, and a pulse duration in the range of 31 μs-183 μs.

The treatment of epilepsy and Parkinson's disease by stimulating the AN of the thalamus 50 and VIM of the thalamus 50, respectively, is by way of example only. Other regions of the thalamus 50 coordinate movement, and thus may be stimulated with different stimulation parameter sets to treat epilepsy and Parkinson's disease.

Furthermore, target sites other than the STN 54 and the thalamus 50 may be stimulated to treat epilepsy or Parkinson's disease. For example, in addition to stimulating the STN 54, the pedunculopontine (PPN) (not shown) may be stimulated with a pulsed electrical waveform having a frequency of 25 Hz and a pulse duration of 60 μs. Additional target sites include the internal segment of the globus pallidus (GPi) 58, which can be stimulated with a pulsed electrical waveform having a frequency in the range of 130 Hz-180 Hz and a pulse duration of 210 μs, or the Zona incerta (ZI) nerve fibers 60, which can be stimulated with a pulsed electrical waveform having a frequency of 150 Hz and a pulse duration of 80 μs. Notably, like the thalamus 50, the GPi 58 is relatively large, and therefore, respective subnuclei (not shown) of the GPi may be concurrently stimulated with multiple pulsed electrical waveforms.

Furthermore, although movement disorders, such as epilepsy and Parkinson's disease, have been described, other dysfunctions can be treated by concurrently delivering multiple pulsed electrical waveforms having different stimulation parameters to multiple target sites via a single stimulation lead.

For example, a stimulation lead may be situated such that the electrodes are adjacent to both the ventral capsule/ventral striatum (VC/VS) (not shown) and the subgenual cortex (not shown), which regions can both be stimulated to treat depression. Thus, both the VC/VS and the subgenual cortex can be concurrently stimulated using the same stimulation lead by generating and concurrently delivering, in multiple timing channels, pulsed electrical waveforms to the specific electrodes that are adjacent the VC/VS and the subgenual cortex. In one exemplary method, the VC/VS is stimulated with a pulsed electrical waveform having a frequency of 127 Hz and a pulse duration of 115 μs, while the subgenual cortex is stimulated with a pulsed electrical waveform having a frequency of 130 Hz and a pulse duration of 90 μs.

In a special case, pulsed electrical waveforms having different pulse durations may be delivered via a single stimulation lead to different target sites respectively formed of nerve cell bodies and nerve fibers, with the pulsed electrical waveform or waveforms with the relatively long pulse duration being delivered to the target site or sites formed of nerve cell bodies, and the pulsed electrical waveform or waveforms with the relatively short pulse duration being delivered to the target site or sites form of nerve fibers. As one example, nerve cell bodies in the STN can be targeted with a pulsed electrical waveform having a relatively long pulse duration, and ZI nerve fibers can be targeted with a pulsed electrical waveform having a relatively short pulse duration. As another example, nerve cell bodies in the VIM can be targeted with a pulsed electrical waveform having a relatively long pulse duration, and the nerve fibers entering the VIM can be targeted with a pulsed electrical waveform having a relatively long pulse duration.

Although particular embodiments of the present inventions have been shown and described, it will be understood that it is not intended to limit the present invention to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed as new and desired to be protected by Letters Patent of the United States is:

1. A method for treating a patient having a dysfunction using a stimulation lead implanted within the brain of the patient, the implanted stimulation lead carrying a plurality of electrodes located adjacent a respective plurality of regions of the brain, the method comprising:
delivering a first electrical waveform at a first pulse frequency to a first region of the brain using a first electrode set comprising at least one of the electrodes of the implanted stimulation lead; and
concurrently delivering a second electrical waveform at a second pulse frequency to a second region of the brain using a second electrode set comprising at least one of the electrodes of the implanted stimulation lead, wherein the first and second electrode sets are not identical, but at least one of the electrodes is in both the first electrode set and the second electrode set, thereby stimulating the brain to treat the dysfunction.

2. The method of claim 1, wherein the dysfunction is Parkinson's disease.

3. The method of claim 2, wherein the first region of the brain comprises a ventralis intermedius of the thalamus and the second region of the brain comprises a subthalamic nucleus.

4. The method of claim 2, wherein the first region of the brain comprises a pedunculopontine and the second region of the brain comprises a subthalamic nucleus.

5. The method of claim 2, wherein the first region of the brain comprises zona incerta nerve fibers and the second region of the brain comprises a subthalamic nucleus.

6. The method of claim 1, wherein the dysfunction is epilepsy.

7. The method of claim 6, wherein the first region of the brain comprises an anterior nucleus of the thalamus and the second region of the brain comprises a subthalamic nucleus.

8. The method of claim 1, wherein the first region of the brain is formed of nerve cell bodies and the second region of the brain is formed of nerve fibers.

9. The method of claim 1, wherein the implanted stimulation lead is coupled to a single implantable pulse generator.

10. The method of claim 1, wherein the plurality of electrodes are in line along the stimulation lead.

11. The method of claim 1, wherein the first pulse frequency and the second pulse frequency are different.

12. The method of claim 1, wherein the first and second electrical waveforms have different pulse durations.

13. The method of claim 1, further comprising implanting the stimulation lead within the brain of the patient.

14. An implantable pulse generator coupleable to an implantable stimulation lead carrying a plurality of electrodes, the implantable pulse generator comprising:
a processor configured and arranged to:
generate a first electrical waveform at a first pulse frequency and direct delivery of the first electrical waveform to a first electrode set containing at least one of the electrodes of the implantable stimulation lead; and
concurrently generate a second electrical waveform at a second pulse frequency and direct delivery of the second electrical waveform to a second electrode set containing at least one of the electrodes of the implantable stimulation lead, wherein at least one of the electrodes is in both the first electrode set and the second electrode set.

15. The implantable pulse generator of claim 14, wherein the first pulse frequency and the second pulse frequency are different.

16. The implantable pulse generator of claim 14, wherein the first and second electrical waveforms have different pulse durations.

17. An electrical stimulation system, comprising:
an implantable stimulation lead comprising a plurality of electrodes; and
an implantable pulse generator coupled to the implantable stimulation lead, the implantable pulse generator comprising a processor configured and arranged to:
generate a first electrical waveform at a first pulse frequency and direct delivery of the first electrical waveform to a first electrode set containing at least one of the electrodes of the implantable stimulation lead; and
concurrently generate a second electrical waveform at a second pulse frequency and direct delivery of the second electrical waveform to a second electrode set containing at least one of the electrodes of the implantable stimulation lead, wherein at least one of the electrodes is in both the first electrode set and the second electrode set.

18. The electrical stimulation system of claim 17, wherein the first pulse frequency and the second pulse frequency are different.

19. The electrical stimulation system of claim 17, wherein the first and second electrical waveforms have different pulse durations.

20. The electrical stimulation system of claim 17, wherein individual pulses of the first electrical waveform do not temporally overlap with individual pulses of the second electrical waveform.
